(19) United States
(12) Patent Application Publication
(10) Pub. No.: US 2023/0222106 A1
(43) Pub. Date: Jul. 13, 2023

(54) STORING AND PROCESSING LONGITUDINAL DATA SETS

(71) Applicant: GENINCODE PLC, Oxford (GB)

(72) Inventors: Christopher John HODKINSON, Cambridgeshire (GB); Julie Christine BARNES, Herfordshire (GB)

(21) Appl. No.: 17/996,327

(22) PCT Filed: Apr. 14, 2021
(86) PCT No.: PCT/GB2021/050898

§ 371 Date: Oct. 14, 2022

Related U.S. Application Data
(60) Provisional application No. 63/011,486, filed on Apr. 17, 2020.

(51) Int. Cl. G06F 16/215; G06F 16/903; G16H 10/60; G16H 70/60; G06F 21/62

(52) U.S. Cl. CPC G06F 16/215; G06F 16/90344; G16H 10/60; G16H 70/60; G06F 21/6245

(57) ABSTRACT

The invention relates to the processing of a data file containing one or more longitudinal data points relating to a subject. The data file is processed in a manner that reduces the risk of erroneously associating its constituent longitudinal data point(s) with an incorrect subject, or failing to associate the longitudinal data point(s) with previously gathered longitudinal data corresponding to the same subject. The invention has application in many fields including the performance of medical tests, particularly tests for biomarkers such as the ROCA test for CA125. Techniques for securely performing analysis of longitudinal data are also provided.

### BACKGROUND

In the modern world large volumes of data are continuously being gathered, stored, collated, and analysed. The data gathered can take many different forms, one of which is longitudinal data. This is data that is gathered over a period of time, where each data point relates to the same subject. Typically, the time over which the data is gathered is significant; e.g. hours, days, months or even years can separate adjacent data points.

In cases where the time between adjacent data points is significant, changes can occur between the collection of adjacent data points. For example, changes to the data gathering system can occur over this timescale. Such changes could be, for example, software and/or hardware updates, a second party taking over responsibility for data recording from a first party, changes to personnel conducting the data recording, changes within a regulated environment that impact upon data gathering and/or storage, and many others.

Changes such as these can be problematic in the case of longitudinal data sets and can lead to orphaned data points. An orphaned data point is a data point that relates to an already gathered longitudinal data set, but which is not properly associated with this existing longitudinal data set. The orphaned data point may be erroneously treated as the first point of a new longitudinal data set, or it may be erroneously associated with a different longitudinal data set. Both scenarios are undesirable because a typical analysis of longitudinal data involves determining how a quantity changes with time. Orphaned data points can lead to inaccurate results of such analyses.

One particular area where orphaned data points can be of significant detriment is medical diagnostic methods. Such methods may rely on detecting a significant deviation from the norm in a longitudinal data set relating to a particular subject in order to perform an accurate diagnosis of a condition. An orphaned data point can lead to a false positive if associated with the wrong longitudinal data set, or a false negative if analysed in isolation from other prior data points relating to the subject. In the case of a medical diagnostic method, either outcome is detrimental.

It is thus clear that there is a need for a technique for gathering longitudinal data that at least reduces the rate of occurrence of orphaned data points, if not entirely eliminates it.

### SUMMARY OF THE INVENTION

The invention relates to the processing of a data file containing one or more longitudinal data points relating to a subject. The data file is processed in a manner that reduces the risk of erroneously associating its constituent longitudinal data point(s) with an incorrect subject, or failing to associate the longitudinal data point(s) with previously gathered longitudinal data corresponding to the same subject. The invention has application in many fields including the performance of medical tests. The medical tests may be of a type that are performed at least annually, but possibly more regularly than that, and perhaps on an aperiodic (irregular) basis. Techniques for securely performing analysis of longitudinal data are also provided.

The invention has application in any scenario in which it is desirable to ensure the accuracy and completeness of a longitudinal data set, and particularly in scenarios where the time between adjacent points in the longitudinal data set is significant, e.g. days, months, years. The invention has particular application in such scenarios where relatively large numbers of longitudinal data sets are stored in parallel, e.g. hundreds, thousands, tens of thousands, and more, where each longitudinal data set relates to a different subject.

One exemplary scenario in which the invention has application is in the context of the ROCA test provided by the Applicant. The ROCA test is implemented by a ROCA algorithm and assists in the diagnosis of ovarian cancer by providing as an output a risk level indicative of the probability that a subject individual has ovarian cancer. The risk is calculated based on the level of a biomarker in a sample of the individual's blood. Notably, the absolute level is not predictive; rather, it is the change in the level of the biomarker over time that is a good predictor as to the individual's risk of having ovarian cancer.

In order to detect the change in the biomarker level it is necessary to first establish a baseline level for the biomarker, which baseline varies from individual to individual. The baseline is established by gathering longitudinal data for the individual over a suitable time period, e.g. months or years. The time between gathering of adjacent longitudinal data points can be significant, e.g. months or years. It is critical that all data points that have been captured are included in the individual's record because missing data points can lead to the baseline being inaccurate, potentially resulting in an incorrect risk score output by the ROCA algorithm. It is also critical that an individual's baseline is assessed based solely on longitudinal data points that have been gathered in relation to that individual rather than some other individual, as inadvertent inclusion of longitudinal data points from a different individual could also cause the ROCA algorithm to output an incorrect risk score.

In a first aspect, the invention provides a computer-implemented method for importing a data file into a longitudinal data store, the method implemented by a server coupled to a database storing the longitudinal data store, the method comprising: receiving the data file, the data file containing at least one longitudinal data point associated with a subject; parsing the data file to identify a first field indicative of whether a record corresponding to the subject exists in the longitudinal data store; determining, by the server, whether a value stored in the first field indicates that a record corresponding to the subject does not exist in the longitudinal data store, wherein in the affirmative the method further comprises: parsing the data file to identify at least one additional field, the or each additional field being associated with respective quantities that are static across all longitudinal data points for the subject; querying the longitudinal data store to determine whether any records having a value stored in the at least one additional field exist; and in the affirmative, flagging the data file as potentially relating to the or each record identified via the querying.

In a second aspect the invention provides a system comprising a server communicatively coupled to a longitudinal data store, the system configured to perform the method of the first aspect.

In a third aspect the invention provides a computer-readable medium storing instructions that, when executed by a server coupled to a longitudinal data store, cause the server to perform the method of the first aspect.

### BRIEF DESCRIPTION OF THE DRAWINGS

FIG. 1 shows in schematic form a system capable of implementing the present invention, according to an embodiment;

FIG. 2 shows a method for processing a data file containing at least one longitudinal data point, according to an embodiment; and,

FIG. 3 shows a method for performing a data analysis operation, according to an embodiment.

### DETAILED DESCRIPTION

The terms listed below have the indicated meaning within this specification:

Longitudinal data: data that relates to a particular subject and which is captured over a time period, where the time period may be of significant length, e.g. days, weeks, months, years. The time between one pair of adjacent data points is not necessarily equal to the time between another paid of adjacent data points in the same longitudinal data set, and indeed in general the time between adjacent data points is variable.

Longitudinal data store: a collection of longitudinal data points stored in a manner such that it is determinable which subject a given data point relates to.

Subject: any entity about which data can be gathered. Of particular focus in this specification is the case where the subject is a biological organism, particularly a human, but the invention is not limited in this manner. Inanimate objects having an identity that is consistent over time can also be subjects, e.g. components in a computer network, buildings on a street, locations within a geographical area, etc.

Record: an entry in a longitudinal data store containing one or more data points relating to a single subject.

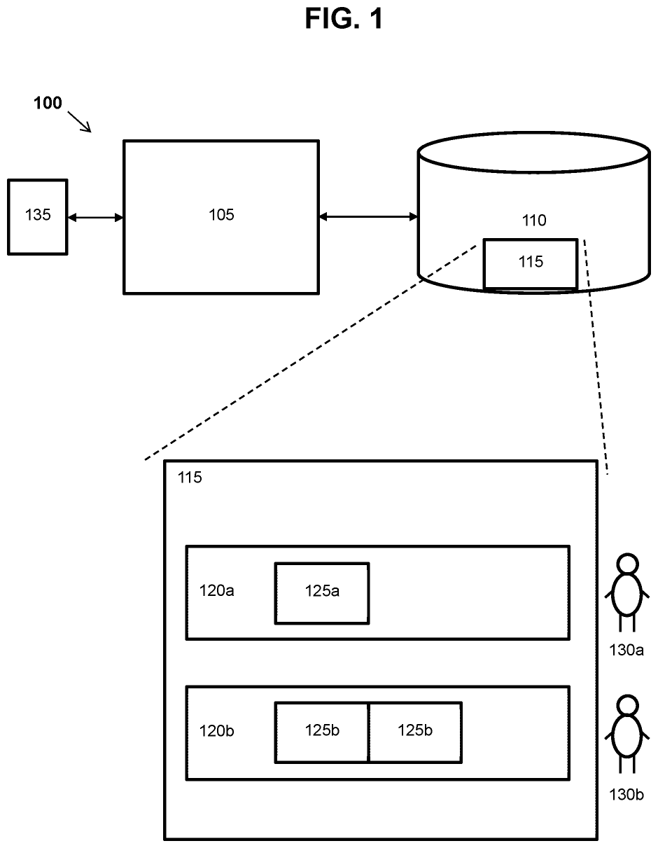

The invention is described below with reference to FIGS. 1, 2 and 3.

FIG. 1 shows a system 100 capable of implementing the present invention. System 100 includes a server 105 coupled to a database 110. Database 110 includes a longitudinal data store 115 that is shown in more detail in the lower portion of FIG. 1. Longitudinal data store 115 can be in any form suitable for electronic storage of data, e.g. an SQL database. Longitudinal data store 115 comprises one or more records 120a, 120b. Each record 120a, 120b contains one or more data points 125a, 125b relating to a single subject 130a, 130b. In this example subjects 130a and 130b are each a person who has undergone a diagnostic test, e.g. a ROCA test as made available by the Applicant, but the invention is not limited in this regard. Reference is made at this point to the definition of ‘subject’ provided above. Data points can be imported into longitudinal data store 115 via a data file in the manner discussed later in this specification. The data file contains at least one data point for importing.

System 100 may further include a data processing device 135 that is communicatively coupled to server 105, e.g. via a WAN like the internet, a LAN, a VPN, or any other network. Data processing device 135 can function to provide new data points to server 105. Data processing device 135 can thus be any electronic device that is capable of transmitting data to server 105, e.g. a desktop or laptop computer, a tablet, a mobile phone, etc. In the case where the subject is a person, data processing device 135 may be operated by a clinician or the subject themselves. Data processing device 135 may gather data about the subject directly, e.g. using one or more embedded sensors, or data processing device 135 may communicatively couple with one or more separate sensors in order to retrieve data points for transmission onward to server 105.

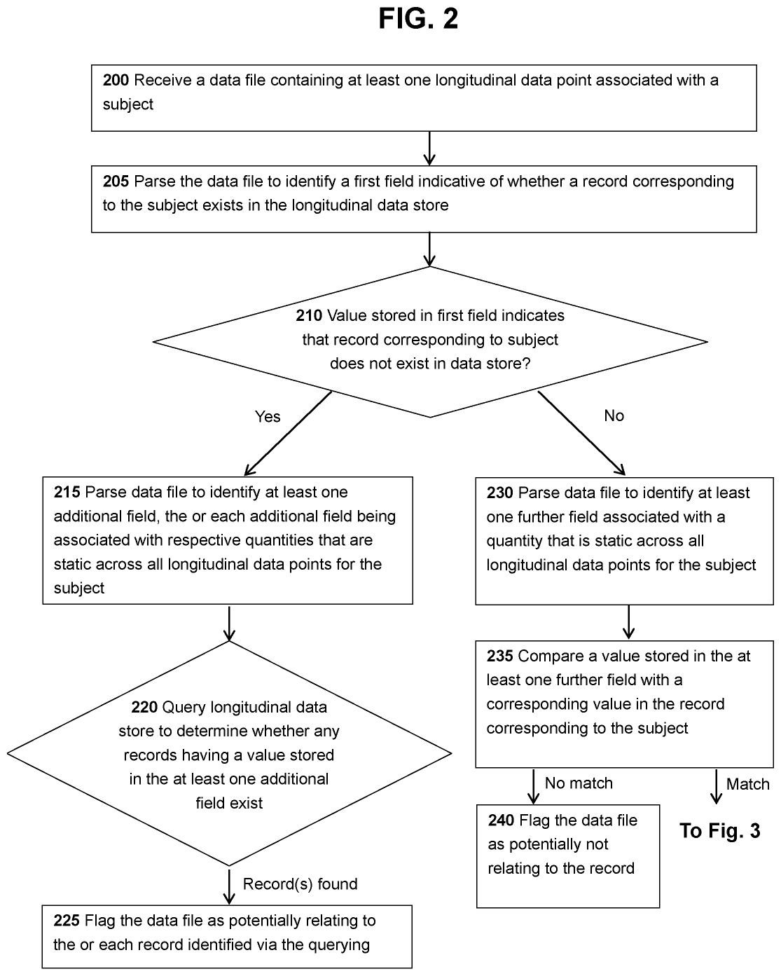

System 100 can be configured to import a data file into longitudinal data store 115. In order to achieve this, system 100 can operate in accordance with the method shown in FIG. 2.

In step 200, server 105 receives the data file, e.g. from data processing device 135. The data file contains at least one longitudinal data point associated with a subject. It should be understood that the term ‘data point’ can encompass a collection of individual items of data, as well as a single item of data. Taking for example the context of a medical diagnostic test, a data point may include a number of different pieces of information relating to a patient, including any combination of: name, address, age, date of birth, date of sample, time of sample, age of sample, location at which the sample was obtained, a system generated unique ID, clinician name, clinic name and clinic location. This list is purely exemplary and it will be appreciated that the invention can be implemented using any items of data, which items will be readily selected by the skilled person according to the particular context in which the invention is to operate.

The data file comprises at least one field, where each field is capable of storing information. For example, a date of birth can be stored in a date format field, a name in a text format field, an age in a number format field, etc. Each data file includes a field that indicates whether a record corresponding to the subject exists in the longitudinal data store. The field could be, for example, a Boolean field that holds the value ‘TRUE’ where the record exists and ‘FALSE’ where the record does not exist, or a text field that holds the character ‘Y’ where the record exists and ‘N’ where the record does not exist. This field is referred to as the ‘presence field’ in the discussion below. These examples are purely illustrative, and many variations will be apparent to a skilled person having the benefit of this specification.

In a perfect system the value stored in the presence field would always accurately capture whether or not the record exists, but of course in any realistic system errors occur which means that this field cannot be absolutely trusted. This may be particularly true where the time between storage of adjacent data points for a given subject is significant, e.g. days, weeks, months, years. The invention thus treats the content of the presence field as an initial indicator as to the existence of the record in the longitudinal data store, but does not rely on this value. Instead, cross-checking is performed, as provided in the follow steps of FIG. 2.

In step 205, server 105 parses the data file to identify the field indicative of whether a record corresponding to the subject exists in the longitudinal data store. This parsing includes identifying the presence field and checking a value held in this field. For example, this can include checking whether the presence field holds ‘TRUE’ or ‘FALSE’, or ‘Y’ or ‘N’, or some other equivalent check.

In step 210, server 105 determines whether a value stored in the first field indicates that a record corresponding to the subject does not exist in the longitudinal data store. For example, server 105 may determine that the presence field holds the value ‘N’ or ‘FALSE’, indicating that a record corresponding to the subject does not exist in the longitudinal data store. On the face of it the data file received in step 200 relates to a subject for which no data has been gathered to date, i.e. the at least one longitudinal data point contained within the data file represents the first data point, or first series of data points, gathered in relation to the subject. The invention takes this only as an indicator, and does not rely upon the value stored in the presence field. Additional steps are performed, as described below, in order to verify that the value stored in the presence field is either correct or incorrect. The invention is therefore particularly suited for use in scenarios where it is very important to ensure that a longitudinal data set is complete.

In the case where the determination of step 210 is in the negative, the method proceeds to step 215. In step 215, server 105 parses the data file to identify at least one additional field, the or each additional field being associated with respective quantities that are static across all longitudinal data points for the subject. In the case where the subject is a person, the at least one additional field can be, for example, any one or more of: first name, last name, full name (i.e. first and last name, with optional middle name(s)), date of birth and a unique identifier assigned to the person during an enrolment process. Other suitable static fields will be apparent to a skilled person having the benefit of this disclosure. In the case of subjects that are not people, suitable static fields will be apparent to a skilled person having the benefit of this disclosure.

Following step 215, in step 220 server 105 queries longitudinal data store 115 to determine whether any records having a value stored in the at least one additional field exist. This may be performed via a lookup operation where a query having a value extracted from the or each additional field is created and submitted to the longitudinal data store 115. For example, where the additional fields are last name and date of birth, a query of the form {last name, date of birth} may be submitted to longitudinal data store 115.

In the case where the result of the querying in step 220 is in the affirmative, i.e. at least one record is found in the longitudinal data store 115 that matches the query, the method moves to step 225 and flags the data file received in step 200 as potentially relating to the or each record identified via the querying. Flagging the data file may involve, for example, setting a value associated with the data file as indicating that one or more duplicates may exist. Here, a duplicate is understood as referring to a first record that concerns the same subject as a second, different record, where there is no link between the first and second records recorded in longitudinal data store 115. A user may be alerted that a duplicate record exists, e.g. by a human-readable message being sent and/or displayed, or similar. For example, in the case of a clinical test such as the ROCA test, upon identification of a duplicate, an electronic message such as an email may be sent to a data processing device from which the data file was received. The electronic message may request validation of the value stored in the presence field. For example, in the case of the ROCA test, upon detection of a duplicate an electronic message may be sent to a device of a clinician to request confirmation that the subject has indeed not had a ROCA test performed previously.

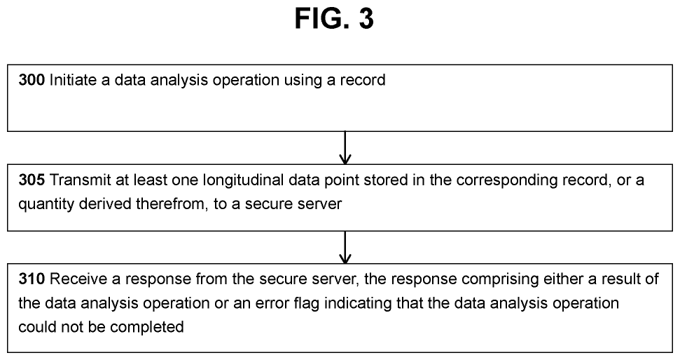

Preferably, in the case where a duplicate is identifier, server 105 is configured to prevent further processing of the data file until the flag applied in step 225 has been removed. Further processing may include using the data file or a part therefore in a clinical test, e.g. the ROCA test. The flag may be removed by a system administrator or other such authorised entity. The flag may be removed based on feedback provided by the provider of the data file, e.g. via an electronic message exchange or other such interaction. The feedback may indicate that the data file received in step 200 relates to one or more records identified in the querying of step 200. Server 105 may be configured to store at least one longitudinal data point from the data file in the one or more records identified in the querying of step 200 so as to form one or more updated records. In this way the orphaned data file may be reunited with the correct record or records, preserving the integrity of the longitudinal data. The method of FIG. 3 may then be performed using the one or more updated records, as following the update the record(s) will contain complete longitudinal data that is ready for use in a further process, e.g. a medical diagnostic test such as the ROCA test.

Returning now to step 210, in the case where the value stored in the first field indicates that a record does exist in the data store 115 corresponding to the subject, the method proceeds to step 230. For example, server 105 may determine that the presence field holds the value ‘Y’ or ‘TRUE’, indicating that a record corresponding to the subject does exist in the longitudinal data store.

In step 230, the data file is parsed to identify at least one further field associated with a quantity that is static across all longitudinal data points for the subject. This is performed in substance in the same manner as step 215 and so is not described in detail again here. In the case where the subject is a person, the at least one further field can be, for example, any one or more of: first name, last name, full name (i.e. first and last name, with optional middle name(s)), date of birth and a unique identifier assigned to the person during an enrolment process. Other suitable static fields will be apparent to a skilled person having the benefit of this disclosure. In the case of subjects that are not people, suitable static fields will be apparent to a skilled person having the benefit of this disclosure.

In step 235, server 105 compares a value stored in the at least one further field with a corresponding value in the record corresponding to the subject. The record corresponding to the subject may be identified in the data file, e.g. using a unique identifier associated with the record. In the case where there is a match, the method may proceed to FIG. 3 to update the record with at least one longitudinal data point from the data file and initiate a data analysis operation using the updated record. This is because in the case of a match it is considered sufficiently likely that the data file does indeed relate to the record that it suggests it is related to, such that adding of data point(s) from the data file to the record is approved.

In the case where there is no match, the method proceeds to step 240. In step 240, the data file is flagged as potentially not relating to the record that it purports to be related to. Flagging the data file may involve, for example, setting a value associated with the data file as indicating that the data file may be incorrectly associated with a particular record. Remedial action may be taken to either confirm that the identified record is indeed correct, or to find the correct record to associate with the data file. The remedial action may include sending an electronic message to a clinician device requesting review of the record associated with the data file.

A method by which a record is processed is now described with reference to FIG. 3. In step 300, a data analysis operation is initiated using a record. The record may be an updated record, i.e. a record that has had one or more longitudinal data points from the data file added to it following the process of FIG. 2. The record may be a new record that includes only data point(s) from the data file.

The data analysis operation may be any data analysis operation that involves longitudinal data. The data analysis operation may be a medical diagnostic test, for example the ROCA test. It will be appreciated that accuracy of the data analysis operation may be improved by use of longitudinal data that has been pre-processed according to the method of FIG. 2.

Initiation of the data analysis operation preferably involves optional steps 305 and 310. In step 305, server 105 transmits at least one longitudinal data point stored in the record, or a quantity derived therefrom, to a secure server. The secure server is separate from sever 105 in the sense that the secure server is administered by an entity that is different from the entity administering server 105. The entity administering server 105 therefore cannot gain access to the secure server, meaning that the operations performed by the secure server cannot be observed by the entity administering server 105. This is advantageous in the scenario where details of the data analysis operation, e.g. particulars of an algorithm used, re confidential. It is also advantageous in the scenario where it is imperative that aspects of the data analysis operation, e.g. input parameters into an algorithm, must only be set and adjusted by an authorised person.

In step 310, server 105 receives a response from the secure server. The response comprises either a result of the data analysis operation or an error flag indicating that the data analysis operation could not be completed. In the case where the data analysis operation is a medical diagnostic test, the result may be a result of the diagnostic test, e.g. a risk score for a subject having a particular medical condition. The result may be a value indicating the subject's risk of having ovarian cancer, e.g. as calculated by the ROCA test.

Steps 305 and 310 may be implemented as an application programming interface (API) call and response.

Additional security steps may be put in place between the interaction of the secure server and server 105. Upon receipt of a request received from server 105, the secure server may check an IP address of server 105 against an IP address whitelist. The IP address whitelist may define one or more IP addresses or IP address range(s) that are considered trusted, from which the secure server will accept requests to process longitudinal data or quantities derived from longitudinal data.

In the case where the IP address of server 105 is found on the whitelist, the secure server may perform the data analysis operation using the longitudinal data point(s) and/or quantities derived therefrom and provide a result to server 105. In the case where the IP address of server 105 is not found on the whitelist, secure server may transmit an error message to server 105 indicating permission for performing the data analysis operation is denied.

Alternatively or additionally, the secure server may validate a token transmitted by server 105 with the at least one longitudinal data point or the quantity derived therefrom. The token may be issued to server 105 by a token issuing server. In the case where the token is successfully validated by the secure server, the secure server may perform the data analysis operation using the longitudinal data point(s) and/or quantities derived therefrom and provide a result to server 105. In the case where validation of the token fails, secure server may transmit an error message to server 105 indicating permission for performing the data analysis operation is denied.

It will be appreciated from the foregoing that the invention is operable to validate longitudinal data in a manner that minimises the risk of longitudinal data points being associated with incorrect records in a database. This can advantageously lead to improvements in onward processing involving the record such as medical diagnostic tests with increased confidence in the output.

1. A computer-implemented method for importing a data file into a longitudinal data store for medical test data, the method implemented by a server coupled to a database storing the longitudinal data store, the method comprising:
receiving the data file, the data file containing at least one longitudinal data point for a medical test result associated with a subject;
parsing the data file to identify a first field indicative of whether a record corresponding to the subject exists in the longitudinal data store;
determining, by the server, whether a value stored in the first field indicates that a record corresponding to the subject does not exist in the longitudinal data store, wherein in the affirmative the method further comprises:
parsing the data file to identify at least one additional field, the or each additional field being associated with respective quantities that are static across all longitudinal data points for the subject;
querying the longitudinal data store to determine whether any records having a value stored in the at least one additional field exist; and,
in the affirmative, flagging the data file as potentially relating to the or each record identified via the querying.

2. The computer-implemented method of claim 1, wherein the subject is a person and the at least one additional field comprises a field containing a name of the person and a field containing a data of birth of the person.

3. The computer-implemented method of claim 1, wherein the subject is a person and the at least one additional field comprises a field containing a unique identifier assigned to the person during an enrolment process.

4. The computer-implemented method of claim 1, further comprising:
transmitting an electronic message to a data processing device from which the data file was received, the electronic message requesting validation of the value stored in the first field.

5. The computer-implemented method of claim 1, further comprising:
receiving a message indicating that the data file relates to a corresponding record identified in the querying; and,
storing the at least one longitudinal data point in the corresponding record.

6. The computer-implemented method of claim 5, further comprising:
initiating a data analysis operation using the corresponding record.

7. The computer-implemented method of claim 6, wherein the data analysis operation is a medical diagnostic method.

8. The computer-implemented method of claim 6, wherein initiating the data analysis operation comprises:
transmitting at least one longitudinal data point stored in the corresponding record, or a quantity derived therefrom, to a secure server; and,
receiving a response from the secure server, the response comprising either a result of the data analysis operation or an error flag indicating that the data analysis operation could not be completed.

9. The computer-implemented method of claim 8, further comprising:
checking, by the secure server, an IP address of the server against an IP address whitelist; and
transmitting an error message indicating permission for performing the data analysis operation is denied in the case where the IP address of the server is not found on the IP whitelist.

10. The computer-implemented method of claim 8, further comprising:
validating, by the secure server, a token transmitted with the at least one longitudinal data point or the quantity derived therefrom; and,
transmitting an error flag indicating permission for performing the data analysis operation is denied in the case where the token cannot be validated.

11. The computer-implemented method of claim 1, wherein in the case where the value stored in the first field indicates that a record corresponding to the subject does exist in the longitudinal data store, the method further comprises:
parsing the data file to identify at least one further field associated with a quantity that is static across all longitudinal data points for the subject; and,
comparing a value stored in the at least one further field with a corresponding value in the record corresponding to the subject,
wherein, in the event the comparing indicates that the value stored in at least one further field does not match the corresponding value in the record, the method further comprises flagging the data file as potentially not relating to the record.

12. The computer-implemented method of claim 11, wherein the subject is a person and the at least one further field comprises a field containing a name of the person and a field containing a data of birth of the person.

13. The computer-implemented method of claim 11, wherein the subject is a person and the at least one further field comprises a field containing a unique identifier assigned to the person during an enrolment process.

14. The computer-implemented method of claim 1, wherein the medical test data comprises the result of a periodic test taken at least annually.

15. The computer-implemented method of claim 1, wherein the medical test is for one or more biomarkers, preferably from a blood sample.

16. The computer-implemented method of claim 1, wherein the medical test is for the diagnosis of cancer.

17. The computer-implemented method of claim 1, wherein the medical test is a ROCA test for the biomarker CA125.

18. A system comprising a server communicatively coupled to a longitudinal data store for medical test data, the system configured to perform the method of claim 1.

19. A computer readable medium storing instructions that, when executed by a server coupled to a longitudinal data store for medical test data, cause the server to perform the method of claim 1.
